(19) United States
(12) Patent Application Publication
(10) Pub. No.: US 2022/0226400 A1
(43) Pub. Date: Jul. 21, 2022

(54) Detection, Treatment, and Monitoring of Microbiome-Mediated Cholesterol Homeostasis

(71) Applicant: McPharma Biotech Inc., Carberry (CA)

(72) Inventors: Derek McLaren, Carberry (CA); Earl McLaren, Carberry (CA)

(21) Appl. No.: 17/613,852

(22) PCT Filed: Apr. 22, 2020
(86) PCT No.: PCT/CA2020/050531

§ 371 Date: Nov. 23, 2021

Related U.S. Application Data
(60) Provisional application No. 62/857,479, filed on Jun. 5, 2019.
(60) Provisional application No. 62/867,369, filed on Jun. 27, 2019.

(51) Int. Cl. A61K 35/747; C12Q 1/689; C12Q 1/6869; A61K 31/718; A61K 31/736; A61K 31/198; A61K 35/745; A61K 35/741; A61K 35/742; A61K 31/702; A61P 3/06

(52) U.S. Cl. CPC A61K 35/747; C12Q 1/689; C12Q 1/6869; A61K 31/718; A61K 31/736; C12Q 2600/112; A61K 35/745; A61K 35/741; A61K 35/742; A61K 31/702; A61P 3/06; A61K 31/198

(57) ABSTRACT

Elucidating the relationship between specific intestinal bacteria, dietary intake, and the health of the host remains a primary goal for gut microbiome research. Prebiotics, substrates that are selectively used by microorganisms to promote the health of the host, present an appealing therapeutic option. We performed correlation analysis to identify relationships between health parameters and populations of gut bacteria in participants from a randomized, placebo-controlled clinical trial testing the effects of a prebiotic digestion resistant potato starch (DRS; MSPrebiotic®). This study focused on the abundance of Parasutterella (phylum Proteobacteria), which tended to increase in the gut microbiome of individuals consuming DRS. Increases in Parasutterella were correlated with reductions in low-density lipoprotein (LDL) levels in participants consuming DRS but not placebo. Segregating DRS-consuming individuals based on whether LDL levels decreased revealed that DRS-consuming individuals who displayed improved LDL levels had significantly higher baseline levels of Parasutterella. Taken together, our analyses suggest that DRS may help improve LDL levels depending on the initial ecological composition of an individual's gut microbiome.

The instant application claims the benefit of U.S. Provisional Patent Application 62/857,479, filed Jun. 5, 2019 and entitled “DETECTION, TREATMENT, AND MONITORING OF MICROBIOME-MEDIATED CHOLESTEROL HOMEOSTASIS”, the entire contents of which are incorporated herein by reference.

The instant application also claims the benefit of U.S. Provisional Patent Application 62/867,369, filed Jun. 27, 2019 and entitled “DETECTION, TREATMENT, AND MONITORING OF MICROBIOME-MEDIATED CHOLESTEROL HOMEOSTASIS”, the entire contents of which are incorporated herein by reference.

### BACKGROUND OF THE INVENTION

The ecosystem of microbes in the human intestines, often referred to as the gut microbiome, influences a wide range of physiological processes and methods to manipulate these connections are actively being investigated (Cani. 2018). Prebiotics stimulate the growth of certain populations of beneficial microbes and therefore offer a strategy to favorably alter the gut microbiome (Gibson et al. 2017). Prebiotic consumption can positively affect the physiology of the host as well as the microbiome (Alfa et al. 2018A, Alfa et al. 2018B), motivating further investigation into the potential health benefits of prebiotics. While the relationships between specific gut microorganisms, dietary intake, and host health outcomes are broadly applicable in principle, dietary interventions to promote host health outcomes may have varying effects depending on the baseline composition of the host's gut microbiome. This reflects both the promise and the challenge of capitalizing on personalized nutrition.

We previously conducted a clinical trial examining the effects of the prebiotic MSPrebiotic® digestion resistant starch (DRS) on the microbiome and various health parameters in two age groups, those 30-50 years old and those 70 years of age or older (Alfa et al. 2018A, Alfa et al. 2018B). We demonstrated that DRS led to significant increases in Bifidobacterium, reductions in Escherichia, and enhanced butyrate production (Alfa et al. 2018B), and improvements in blood glucose, insulin, and insulin resistance (Alfa et al. 2018A) that were correlated with reductions in the abundance of Sporacetigenium (Bush and Alfa. 2018).

The connections between starch-fermenting Bifidobacterium, butyrate-producing members of the phylum Firmicutes, and health outcomes have been well-documented (De Vuyst and Leroy. 2011) but little research has been done to establish connections between other, more obscure bacteria in the gut microbiome. Furthermore, the role of Proteobacteria in the gut microbiome remains ambiguous, with evidence supporting both healthy (Scaldaferri et al. 2016) and pathogenic relationships (Gomes et al. 2016). Probiotic supplementation with the proteobacteria genus Parasutterella reduced bile acids, increased taurine in the gut, and enhanced liver expression of Cyp7a1, which encodes the rate-limiting enzyme responsible for the conversion of cholesterol to cholic acid (Ju et al. 2019). The objective of this study was to explore connections between Parasutterella (phylum Proteobacteria) and various metabolic markers in DRS-consuming individuals.

### SUMMARY OF THE INVENTION

According to an aspect of the innovation, there is provided a method for determining efficacy of a microbiome modulating treatment for high low-density lipid (LDL) cholesterol levels in an individual with dyslipidemia or at risk of developing dyslipidemia, said method comprising:

Detecting Parasutterella levels in a first gut microbiome sample from the individual at a first time point;

Administering to the individual a microbiome modulating treatment on a dosage regimen for a suitable period of time;

Following the suitable period of time, obtaining a second gut microbiome sample from the individual;

Detecting Parasutterella levels in the second sample; and

Comparing Parasutterella levels in the second gut microbiome sample to Parasutterella levels in the first gut microbiome sample,

Wherein if the Parasutterella levels are higher than in the first sample, continuing the dosage regimen for the individual.

In some embodiments, at the first and second time points, LDL cholesterol or at least one other lipid marker is measured in the individual and these two measures are also compared.

According to another aspect of the invention, there is provided a method for determining efficacy of a microbiome modulating treatment for high LDL levels in an individual at risk of developing or who has developed or who has dyslipidemia, said method comprising:

Detecting Parasutterella levels in a first gut microbiome sample from the individual at a first time point;

Determining a level of at least one lipid marker at a first time point;

Administering to the individual a microbiome modulating treatment on a dosage regimen for a suitable period of time;

Following the suitable period of time, obtaining a second gut microbiome sample from the individual;

Detecting Parasutterella levels in the second sample;

Determining a level of the at least one lipid marker of the individual at the second time point;

Comparing Parasutterella levels in the second gut microbiome sample to Parasutterella levels in the first gut microbiome sample; and

Comparing the first lipid marker measurement to the second lipid marker measurement;

Wherein if the Parasutterella levels are higher than in the first sample, and the second lipid marker measurement is improved when compared to the first lipid marker measurement, continuing the dosage regimen for the individual.

As will be appreciated by one of skill in the art, the marker is improved in that the new value is associated with a better or positive or more desirable health outcome. For example, for LDL-cholesterol, improvement would be a lower LDL-cholesterol value.

In some embodiments, the gut microbiome sample, for example but by no means limited to a stool or fecal sample or colonic contents, whether sampled in situ or via intervention.

In some embodiments, the treatment is a microbiome therapy, that is, a treatment that is known to or expected to alter the microbiome of the individual. Examples of microbiome therapies are discussed herein and other examples will be readily apparent to one of skill in the art.

According to an aspect of the invention, there is provided a method for reducing low-density lipid (LDL) cholesterol levels in an individual in need of such treatment comprising:

Detecting Parasutterella levels in a first gut microbiome sample from the individual at a first time point;

If the Parasutterella levels correspond to an effective amount of Parasutterella, administering to the individual a microbiome modulating treatment on a dosage regimen for a suitable period of time.

In some embodiments, following the suitable period of time, at least one lipid marker measurement is taken.

In some embodiments, following the suitable period of time: obtaining a second gut microbiome sample from the individual; detecting Parasutterella levels in the second sample; and comparing Parasutterella levels in the second gut microbiome sample to Parasutterella levels in the first gut microbiome sample, wherein if the Parasutterella levels are higher than in the first sample, continuing the dosage regimen for the individual.

The individual in need of such treatment may be an individual with dyslipidemia or at risk of developing dyslipidemia.

As discussed herein, “an effective amount of Parasutterella” is an amount such that the individual is a “responder” to the microbiome modulating treatment, as discussed herein.

According to another aspect of the invention, there is provided a method for converting a Parasutterella microbiome modulating treatment non-responder to a Parasutterella microbiome-modulating responder comprising:

Administering a Parasutterella increasing compound to an individual who has gut microbiome levels of Parasutterella below a responder threshold level on a dosage schedule or regimen.

In some embodiments of the invention, the administration of the Parasutterella increasing compound continues until the gut microbiome levels of Parasutterella of the individual are above the responder threshold level, that is, until the individual has an effective amount of gut microbiome Parasutterella.

In some embodiments, a first gut microbiome sample is taken from the individual prior to administering the Parasutterella-increasing compound.

In some embodiments, a second gut microbiome sample is taken from the individual following administration, for example, following a suitable period of time on the dosage schedule or regimen.

According to another aspect of the invention, there is provided a method for detecting the signature of an unusually favourable gut microbiome that is correlated with beneficial cholesterol metabolism in an individual compromising the monitoring of Parasutterella levels and predicting the efficacy of microbiome therapies if Parasutterella is present.

As will be apparent to one of skill in the art, Parasutterella may be detected in a sample by a variety of means illustrated by the examples below.

In some embodiments of the invention, Parasutterella is detected by directed 16S V4 ribosomal subunit amplification (for example, by Real-Time Polymerase Chain Reaction; RT-PCR, or Quantitative PCR; qPCR) of Parasutterella using the abundance of Blautia or some other common commensal bacteria unrelated to cholesterol homeostasis as the reference value. As will be apparent to one of skill in the art, Blautia is both common (found in most microbiomes) and abundant (making up a large proportion of each microbiome), and accordingly is suitable to be used as an internal control. However, other suitable candidates for use as an internal control will be readily apparent to one of skill in the art.

In other embodiments of the invention, Parasutterella is detected by whole microbiome sequencing using the 16S V4 ribosomal subunit and/or other relevant regions.

In another embodiment of the invention, Parasutterella is detected by shotgun metagenome sequencing, other unbiased genomic approaches, or any method that reports the proportional representation of Parasutterella in the microbiome.

In some embodiments of the invention, the individual has dyslipidemia, as defined by LDL cholesterol levels of 130 mg/dL (3.4 mmol/L) or higher, and/or a ratio of LDL to high density lipoprotein (HDL) of 2.5 or higher, and/or a ratio of HDL to LDL of 0.4 or less, and/or triglycerides (TG) 150 mg/dL (1.7 mmol/L) or higher, and/or a ratio of TG to HDL of 3 or higher, and/or a total cholesterol (C) level of 200 mg/dL (5.2 mmol/L) or higher, and or a ratio of C to HDL of 4 or higher, and/or a non-HDL cholesterol level of 130 mg/dL (3.3 mmol/L) or higher, and/or as determined by a physician.

In another embodiment of the invention, the individual is at risk of developing cardiovascular disease, and/or coronary heart disease, and/or metabolic syndrome, and/or atherosclerosis, and/or stroke, and/or Type 2 Diabetes (T2D), and/or peripheral vascular disease, and/or hypertension due to family history or lifestyle factors.

In another embodiment of the invention, the individual has been diagnosed with or is suspected of having cardiovascular disease, and/or coronary heart disease, and/or metabolic syndrome, and/or atherosclerosis, and/or stroke, and/or T2D, and/or peripheral vascular disease, and/or hypertension.

In some embodiments of the invention, the microbiome intervention is a prebiotic, administered daily or as needed, for as long as the lipid markers continue to show improvement compared to baseline levels.

As discussed herein, the prebiotic microbiome therapy may be resistant potato starch or a resistant potato starch product, delivered daily or as needed, for as long as the lipid markers continue to show improvement compared to baseline levels.

As discussed herein, the effective amount may be for example 2 to 40 g, 2 to 30 g, 2 to 20 g, 5 to 40 g, 5 to 30 g, 5 to 20 g, or 10 to 20 g of resistant potato starch.

In another embodiment of the invention, the microbiome intervention is a probiotic, administered daily or as needed, for as long as the lipid markers continue to show improvement compared to baseline levels.

In another embodiment of the invention, the microbiome intervention may involve probiotic supplementation with Parasutterella, administered daily or as needed, if baseline Parasutterella levels are suspected of being too low, that is, below the threshold level to constitute an effective amount within the gut microbiome of the individual and for as long as the lipid markers continue to show improvement compared to baseline levels.

In another embodiment of the invention, the microbiome intervention is an antibiotic, administered daily or as needed, for as long as the lipid markers continue to show improvement compared to baseline levels.

In another embodiment of the invention, the microbiome intervention is a combination of prebiotics and/or probiotics and/or antibiotics and/or bacteriophages, administered daily or as needed, for as long as the lipid markers continue to show improvement compared to baseline levels.

In another embodiment of the invention, the microbiome intervention is a combination of Parasutterella probiotic and/or prebiotics and/or other probiotics and/or antibiotics and/or bacteriophages, administered daily or as needed, for as long as the lipid markers continue to show improvement compared to baseline levels.

### BRIEF DESCRIPTION OF THE DRAWINGS

FIG. 1. Simplified view of LDL-related cholesterol transport at the gut. Cholesterol is utilized in the production of Bile Salts by the Liver, which are secreted into the intestines to emulsify and absorb dietary fats. The majority of this cholesterol is returned to the body, including in the form of LDL, and recycled by the liver. A small amount of Cholesterol is lost in the feces.

FIG. 2. Canonical mechanism of probiotic-mediated lowering of cholesterol. Probiotic bacteria, for example Lactobacillus, which may or may not be stimulated by prebiotics, and secrete Bile Salt Hydrolases that cleave Bile Salts to prevent re-uptake cholesterol. This decreases levels of cholesterol, including LDL, and enhances cholesterol efflux.

FIG. 3. Mean change (+/−SEM) in relative abundance for each genus discretely identified in individuals consuming DRS for 12 weeks.

FIG. 4. DRS consumption tended to increase mean levels of Parasutterella by two-fold (p=0.0711) while Parasutterella levels were unchanged in those consuming placebo (+/−SEM, p=0.291).

FIG. 5. Segregation of DRS consumers into those who displayed a decrease in LDL levels (Responders) and those whose levels increased or remained the same (Non-Responders) revealed that mean baseline Parasutterella levels were significantly higher in Responders (+/−SEM, p=0.0389).

FIG. 6. Stimulation of Parasutterella via prebiotic, including Digestion Resistant Starch, leads to reduced LDL levels but the mechanism of action remains to be determined. It is possible that Parasutterella (A) stimulates Lactobacilli or similar bacteria to produce Bile Salt Hydrolases (or related molecules) that, (B) produces or enhances the activity of Bile Salt Hydrolases, (C) stimulates the Liver via unknown metabolites to alter LDL cholesterol, or (D) has effects on LDL levels independent of the possibilities listed here.

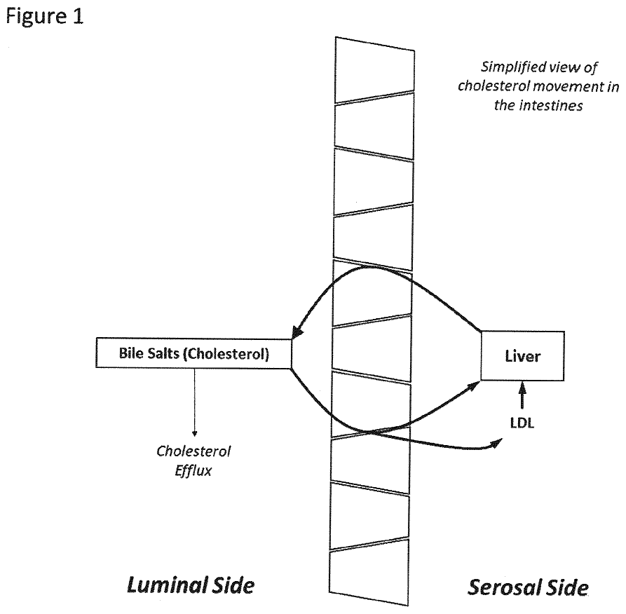

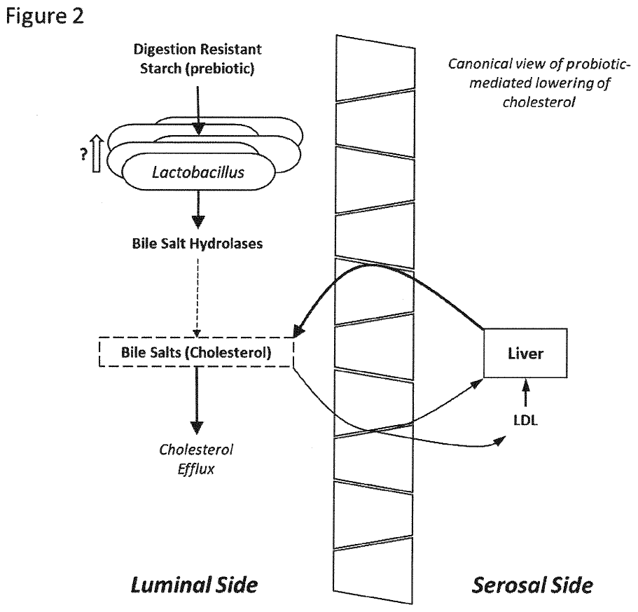

### DESCRIPTION OF THE PREFERRED EMBODIMENTS

Unless defined otherwise, all technical and scientific terms used herein have the same meaning as commonly understood by one of ordinary skill in the art to which the invention belongs. Although any methods and material similar or equivalent to those described herein can be used in the practice or testing of the present invention, the preferred methods and materials are now described. All publications mentioned hereunder are incorporated herein by reference.

We investigated the correlation between cholesterol markers and changes in the microbiome in response to supplementation with prebiotic resistant potato starch (MSPrebiotic®).

MSP Starch Products Inc. manufactures MSPrebiotic® Resistant Potato Starch, an unmodified Type 2 resistant starch (RS2) that is a Solanum tuberosum extract preparation of food grade quality for human and animal application. Resistant potato starch is also referred to as digestion or digestive resistant starch (DRS). While MSPrebiotic®, which contains 7 g of fiber in 10 g of product, is used in the trials and experiments discussed herein, it is important to note that as discussed herein, another suitable resistant potato starch or potato resistant starch or resistant potato starch product, that is, another unmodified RS type 2 potato starch, comprising at least 60% resistant starch or at least 65% resistant starch or at least 70% resistant starch or at least 75% resistant starch or at least 80% resistant starch of total extract or total potato extract may be used. That is, the extract itself may comprise at least 60% resistant starch, at least 65% resistant starch, at least 70% resistant starch, at least 75% resistant starch, or at least 80% resistant starch on a dry weight basis.

In some embodiments, the resistant potato starch is derived or prepared from varieties of potato naturally containing high levels of asparagine and/or aspartate, such as, but not limited to, Russet potatoes.

According to an aspect of the invention, there is provided a method for determining the efficacy of a microbiome modulating treatment for high LDL in an individual at risk of developing or who is developing or who has dyslipidemia, said method comprising:

Detecting Parasutterella levels in a first gut microbiome sample from the individual at a first time point;

Administering to the individual a microbiome modulating treatment on a dosage regimen for a suitable period of time;

Following the suitable period of time, obtaining a second gut microbiome sample from the individual;

Detecting Parasutterella levels in the second sample; and

Comparing Parasutterella levels in the second gut microbiome sample to Parasutterella levels in the first gut microbiome sample,

Wherein if the Parasutterella levels are higher than in the first sample, continuing the dosage regimen for the individual.

In some embodiments, at the first and second time points, LDL cholesterol or at least one other lipid marker is measured in the individual and these two measures are also compared.

According to another aspect of the invention, there is provided a method for determining efficacy of a microbiome modulating treatment for high LDL levels in an individual at risk of developing or who has developed or who has dyslipidemia, said method comprising:

Detecting Parasutterella levels in a first gut microbiome sample from the individual at a first time point;

Determining the level of at least one lipid marker at a first time point;

Administering to the individual a microbiome modulating treatment on a dosage regimen for a suitable period of time;

Following the suitable period of time, obtaining a second gut microbiome sample from the individual;

Detecting Parasutterella levels in the second sample;

Determining the level of at least one lipid marker of the individual at a second time point;

Comparing Parasutterella levels in the second gut microbiome sample to Parasutterella levels in the first gut microbiome sample; and

Comparing the at least one lipid marker measurement at the first time point to the at least one lipid marker measurement at the second time point;

Wherein if the Parasutterella levels are higher in the second sample than in the first sample, and the lipid marker measurement at the second time point is improved when compared to the lipid marker measurement at the first time point, for example, has changed in a manner that is consistent with a positive health outcome, continuing the dosage regimen for the individual.

For example, a change in LDL-cholesterol that is “consistent with a positive health outcome” is a decrease in LDL-cholesterol over time.

According to an aspect of the invention, there is provided a method for reducing low-density lipid (LDL) cholesterol levels in an individual in need of such treatment comprising:

Detecting Parasutterella levels in a first gut microbiome sample from the individual at a first time point;

If the Parasutterella levels correspond to an effective amount of Parasutterella, administering to the individual a microbiome modulating treatment on a dosage regimen for a suitable period of time.

In some embodiments, following the suitable period of time, at least one lipid marker measurement is taken.

In some embodiments, following the suitable period of time: obtaining a second gut microbiome sample from the individual; detecting Parasutterella levels in the second sample; and comparing Parasutterella levels in the second gut microbiome sample to Parasutterella levels in the first gut microbiome sample, wherein if the Parasutterella levels are higher than in the first sample, continuing the dosage regimen for the individual.

The individual in need of such treatment may be an individual with dyslipidemia or at risk of developing dyslipidemia.

As discussed herein, “an effective amount of Parasutterella” is an amount such that the individual is a “responder” to the microbiome modulating treatment, that is, has beneficial cholesterol metabolism that responds to microbiome modulation, as discussed herein. For example, as a result of microbiome modulation, the individual may have enhanced or improved or increased cholesterol metabolism, that is, enhanced or improved or increased microbiome-modulated cholesterol metabolism.

According to another aspect of the invention, there is provided a method for converting a Parasutterella microbiome modulating treatment non-responder to a Parasutterella microbiome-modulating responder comprising:

Administering a Parasutterella increasing compound to an individual who has gut microbiome levels of Parasutterella below a responder threshold level on a dosage schedule or regimen.

In some embodiments of the invention, the administration of the Parasutterella increasing compound continues until the gut microbiome levels of Parasutterella of the individual are above the responder threshold level, that is, until the individual has an effective amount of gut microbiome Parasutterella, that is, until the individual is capable of beneficial cholesterol metabolism, as discussed herein.

In some embodiments, a first gut microbiome sample is taken from the individual prior to administering the Parasutterella-increasing compound.

In some embodiments, a second gut microbiome sample is taken from the individual following administration, for example, following a suitable period of time on the dosage schedule or regimen.

As discussed herein, suitable Parasutterella-increasing compounds include but are by no means limited to compounds known in the art that will effect dietary changes that increase the availability of asparagine, aspartate, alanine, arginine, glycine, leucine, and/or other amino acids and/or other fermentation substrates to Parasutterella in the digestive tract; and antibiotics or bacteriophage that target a bacterium/other bacteria that inhibit or limits the growth of Parasutterella.

It is of note that while it may be more convenient to obtain samples for Parasutterella and the lipid marker(s) or measurement(s) at the same time, this is not a requirement of the invention. That is, the samples do not necessarily need to be taken at exactly the same time but may be taken separately within a reasonable time period and still be considered as having been taken at either the first time point of the second time point as the case may be. For example, taking samples as close as possible will provide greater accuracy for determining trends; however, blood lipid parameters for the individual may be known for example from the last check-up for the individual.

Similarly, the measuring of samples does not need to be done immediately or even by the same institution. That is, means for storing suitable samples for measurement of bacterial levels or lipid markers or other measurements are well-known in the art.

The individual who is at risk of developing dyslipidemia may be at risk based on genetic predisposition, familial history, heredity, lifestyle, and/or one or more dyslipidemia-related parameters being evaluated, for example, low density lipoprotein (LDL) levels, high density lipoprotein (HDL) levels, total cholesterol, total triglycerides, ratios involving LDL, HDL, total cholesterol, and/or triglycerides. As discussed above, the individual may also be an individual who has cardiovascular disease, that is, an individual who has been diagnosed with cardiovascular disease. Similarly, the individual may be an individual who has developed cardiovascular disease, that is, an individual who has recently developed cardiovascular disease and who may or may not have been diagnosed with cardiovascular disease or who is at risk of developing cardiovascular disease.

As discussed herein, we demonstrate a method for detecting and treating individuals with impaired lipid homeostasis who are sensitive to microbiome-targeted therapeutic intervention using a microbiome modulating compound. In some embodiments, the microbiome modulating compound is prebiotic resistant potato starch as defined herein.

In other embodiments, the microbiome modulating compound is selected from the group consisting of: resistant potato starch, probiotic genera, species, and strains; prebiotics supporting growth of probiotic genera, species and strains; resistant starch from corn, tapioca, banana, grains, tubers and the like; resistant starch derived from high asparagine and/or aspartate-containing varieties of potato, corn, tapioca, banana, grains, tubers and the like; fructooligosaccharides; galactooligosaccharides; xylooligosaccharides, mannanoligosaccharides; arabinoxylooligosaccharides; arabinogalactan polysaccharides; galactomannan polysaccharides, asparagine, asparagine-containing peptides or proteins, and carbohydrate-amino acid complexes that contain asparagine; and aspartate, aspartate-containing peptides or proteins, and carbohydrate-amino acid complexes that contain aspartate. As will be appreciated by one of skill in the art, “high” or “elevated” or “increased” in regards the asparagine and/or aspartate levels of a particular resistant starch or resistant starch product is a relative term and refers to a resistant starch or resistant starch product that has higher asparagine and/or aspartate levels than for example one or more comparable resistant starch or resistant starch product from a different source. Similarly, “asparagine-containing peptides or proteins, and carbohydrate-amino acid complexes that contain asparagine” refer to peptides, proteins and/or complexes that contain what one of skill in the art would consider to be elevated levels of asparagine compared to comparable peptides, proteins and/or complexes and , “aspartate-containing peptides or proteins, and carbohydrate-amino acid complexes that contain aspartate” refer to peptides, proteins and/or complexes that contain what one of skill in the art would consider to be elevated levels of aspartate compared to comparable peptides, proteins and/or complexes.

Dietary changes that support the growth of healthy bacteria, including the probiotic bacteria listed above:
Dietary changes that increase the availability of asparagine, aspartate, alanine, arginine, glycine, leucine, and/or other amino acids and/or other fermentation substrates to Parasutterella in the digestive tract.
Antibiotics or bacteriophage that target a bacterium/other bacteria that inhibit or limits the growth of Parasutterella.

The probiotic genera, species and strains may be selected from the group consisting of: Parasutterella; Bifidobacterium; Staphylococcus; Clostridium; Lactobacillus; Prevotella; Barnsiella; and combinations thereof.

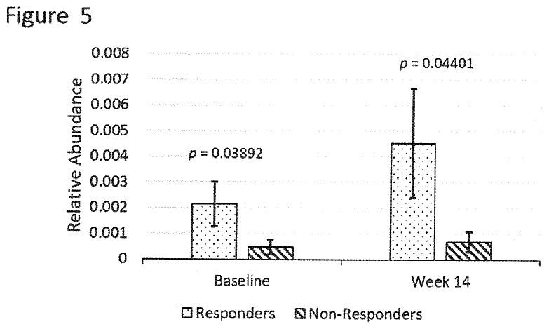

As will be appreciated by one of skill in the art, and as discussed herein, Parasutterella is particularly well-suited for use as a probiotic, that is, for use in increasing Parasutterella levels in an individual who is possibly a non-responder who wishes to be a responder or an individual who wishes to increase their level of beneficial cholesterol metabolism as defined herein. Specifically, it is noted that as shown in FIG. 5, relative abundance of Parasutterella increased in both responders and non-responders without adversely affecting bacterial diversity. Furthermore, the absence of toxin virulence factor (VF)-related genes in the genome of Parasutterella mc1 suggests that Parasutterella is either a commensal or symbiotic member of the microbiome (Ju et al. 2019), and that supplementing with this bacterium in probiotic form will not have obvious adverse effects. The assertion that Parasutterella is a core component of the gut microbiome (Ju et al. 2019) is consistent with our observation that Parasutterella was detectable in 94% of the trial participants. However, not all of these participants had an effective amount of Parasutterella, as discussed herein.

That is, given the beneficial effect on cholesterol metabolism observed when an effective amount of Parasutterella is present in the gut microbiome of an individual, increasing the Parasutterella levels either directly by administering Parasutterella alone or in combination with compounds supporting and/or promoting Parasutterella growth or indirectly by administering compounds that increase Parasutterella growth for example as discussed herein will increase Parasutterella levels to above the responder level in the individual and/or improve or increase the beneficial cholesterol metabolism.

The resistant starch may be RS1, RS2, RS3, RS4, or RS5.

The corn may be high amylose maize;

The grains may be barley, wheat, sorghum, oats or the like.

Examples of suitable high asparagine and/or aspartate-containing resistant starch sources include but are by no means limited to Russet potatoes.

Examples of suitable fructooligosaccharides include but are by no means limited to inulin and inulin-type fructans.

The galactooligosaccharides may be of varying lengths, for example, between 2 and 8 saccharide units, and may include various linkages of galactose for example but by no means limited to β-(1,4),13-(1,6) galactose, and a terminal glucose.

The xylooligosaccharides may be composed of xylose or related C5 sugar oligosaccharides.

The mannanoligosaccharides may be, for example, glucomannanoligosaccharides.

Suitable galactomannan polysaccharides include guar gum.

In other embodiments, the microbiome modulating compound is selected from the group consisting of: resistant potato starch, probiotic genera, species, and strains; prebiotics supporting growth of probiotic genera, species and strains; resistant starch from corn, tapioca, banana, grains, tubers, and the like; resistant starch derived from high asparagine and/or aspartate-containing varieties of potato, corn, tapioca, banana, grains, tubers and the like; fructooligosaccharides; galactooligodsaccharides; xlyooligosaccharides; mannanoligosaccharides; arabinoxyloligosaccharides; arabinogalactan polysaccharides; galactomannan polysaccharides; asparagine, asparagine-containing peptides or proteins, and carbohydrate-amino acid complexes that contain asparagine; aspartate, aspartate-containing peptides or proteins, and carbohydrate-amino acid complexes that contain aspartate; dietary changes that support the growth of probiotic bacteria; dietary changes that increase the availability of asparagine, aspartate, alanine, arginine, glycine, leucine, and/or other amino acids and/or other fermentation substrates to Parasutterella in the digestive tract; and antibiotics that target a bacterium/other bacteria that inhibit the growth of Parasutterella.

In yet other embodiments, the microbiome modulating compound is selected from the group consisting of: resistant potato starch, probiotic genera, species, and strains; prebiotics supporting the growth of probiotic genera, species, and strains; resistant starch from corn, tapioca, banana, grains, tubers, and the like; resistant starch derived from high asparagine and/or aspartate-containing varieties of potato, corn, tapioca, banana, grains, tubers and the like; fructooligosaccharides; galactooligosaccharides; xylooligosaccharides; mannanoligosaccharides; arabinoxylooligosaccharides; arabinogalactan polysaccharides; galactomannan polysaccharides; asparagine, asparagine-containing peptides or proteins, and carbohydrate-amino acid complexes that contain asparagine; aspartate, aspartate-containing peptides or proteins, and carbohydrate-amino acid complexes that contain aspartate; dietary changes that support the growth of probiotic bacteria; dietary treatments that increase the availability of asparagine, aspartate, alanine, arginine, glycine, leucine, and/or other amino acids and/or other fermentation substrates to Parasutterella in the digestive tract; and antibiotics that target a bacterium/other bacteria that inhibit the growth of Parasutterella; mixed plant cell wall fibers; beta-glucans; resistant dextrins; resistant maltodextrins; limit dextrins; polydextrose; alginate; pectin polysaccharides; hydroxypropylmethylcellulose; chitin; chondroitin-containing compounds; and glucosamine-containing compounds.

Preferably, the mixed plant cell wall fibers comprise two or more of the following plant cell wall fibers in varying proportions: cellulose, pectin, lignin, beta-glucan, and arabinoxylan regardless of source.

The beta-glucans may be from cereal, such as for example, mixed-link (1,3, 1,4) beta-glucans from oat, barley, rye, wheat, or the like, or from fungi, for example, yeast, mushroom, and the like, sources.

Resistant dextrins, resistant maltodextrins, and limit dextrins may be from wheat, corn, or other suitable sources. These non-digestible oligosaccharides of glucose molecules are joined by digestible linkages and non-digestible α-1,2 and α-1,3 linkages.

The polydextrose may be highly branched and may contain α- and β-1,2, 1,3, 1,4, and 1,6 linkages, with the 1,6 linkage predominating in the polymer.

The alginate may be β-1,4-D-mannuronic acid and α-1,4-L-guluronic acid organized in homopolymeric compounds of either mannuronate or guluronate, or as heteropolymeric compounds, expressed as mannuronic acid to guluronic acid ratio.

The pectin polysaccharides may have a backbone chain of α-1,4-linked D-galacturonic acid units interrupted by the insertion of 1,2-linked L-rhamnopyranosyl residues in adjacent or alternate positions. These compounds are present in cell walls and intracellular tissues of fruits, vegetables, legumes, and nuts.

Hydroxypropylmethylcellulose, also known as Hypromellose, is a propylene glycol ether of methylcellulose containing methoxyl groups and hydroxypropyl groups.

The chitin may be, for example, from fungi or arthropods. Suitable chondroitin-containing compounds include chondroitin sulfate from animal sources.

Suitable glucosamine-containing compounds include glucosamine sulfate from animal sources.

In some embodiments, the gut microbiome modulating treatment may be or may also include spores from a single strain or specie of bacteria, yeast, or other fungi; bacteriophage or a combination of bacteriophages; or an exogenously produced metabolite or metabolites normally derived from the metabolism of the gut microbiome, also known as postbiotics or parabiotics.

As will be appreciated by one of skill in the art, a cardiovascular disease related parameter as used herein refers to a parameter that is associated with or measured as part of monitoring for cardiovascular disease.

In some embodiments of the invention, the cardiovascular disease related parameter is selected from the group consisting of: low density lipoprotein (LDL) levels, high density lipoprotein (HDL) levels, total cholesterol, total triglycerides, ratios involving LDL, HDL, total cholesterol, and/or triglycerides, systemic and/or tissue-specific inflammation markers for example high sensitivity C-reactive protein (hsCRP), blood pressure, and metabolites for example trimethylamine oxide (TMAO).

As discussed herein, high LDL is generally diagnosed at about 130 mg/dL (3.4 mmol/L) or higher, and/or a ratio of LDL to high density lipoprotein (HDL) of about 2.5 or higher, and/or a ratio of HDL to LDL of about 0.4 or less; high triglycerides (TG) are generally diagnosed at about 150 mg/dL (1.7 mmol/L) or higher, and/or a ratio of TG to HDL of about 3 or higher; high levels of cholesterol are generally diagnosed at total cholesterol (C) levels of about 200 mg/dL (5.2 mmol/L) or higher, and/or a ratio of C to HDL of about 4 or higher, and/or a non-HDL cholesterol level of about 130 mg/dL (3.3 mmol/L) or higher. Inflammation is generally diagnosed at hsCRP levels of about 1.0 mg/L or higher and/or TMAO levels of 6.2 mM or higher, while elevated blood pressure is generally diagnosed at about 120/80 mm Hg or higher.

The period of time may be for example about 1 week, about 2 weeks, about 3 weeks, about 4 weeks, about 5 weeks, about 6 weeks, about 7 weeks, about 8 weeks, about 9 weeks, about 10 weeks, about 11 weeks, about 12 weeks or longer.

Parasutterella levels may be measured using any suitable means known in the art. For example, Parasutterella levels may be measured using real-time polymerase chain reaction (RT-PCR)-based methods; quantitative PCR (qPCR)-based methods; by microbiome sequencing directed at any sequence that defines Parasutterella, including but not limited to the 16S V4 ribosomal subunit sequence; by shotgun metagenomic sequencing; by quantitative fluorescent in situ hybridization (FISH) with probes recognizing sequences that define Parasutterella, including but not limited to the 16S V4 ribosomal subunit sequence; or by antibody or cell-binding based methods.

As will be appreciated by one of skill in the art, the levels of Parasutterella are being measured over time. Consequently, levels of Parasutterella may be determined by direct measurement, using suitable means known in the art, for example, such as those discussed above. Alternatively, the level of Parasutterella in a given sample may be compared to an internal control, for example, using the abundance of Blautia or other common commensal unrelated to lipid metabolism as the reference value. As will be apparent to one of skill in the art, Blautia is common (found in most gut microbiomes) and abundant (making up a large proportion of each microbiome), and accordingly is suitable to be used as an internal control. However, other suitable candidates for use as an internal control will be readily apparent to one of skill in the art. Alternatively, the control may be a non-biological control. Furthermore, as will be appreciated by one of skill in the art, the control does not necessarily need to be repeated with each measurement.

As discussed herein, a “responder” is an individual that has an effective amount of Parasutterella in their gut microbiome, that is, an amount of Parasutterella that is sufficient to respond effectively to the gut microbiome modulating compound and reduce LDL-cholesterol which is defined herein as beneficial cholesterol metabolism. For example, as a result of microbiome modulation, the individual may have enhanced or improved or increased cholesterol metabolism, that is, enhanced or improved or increased microbiome-modulated cholesterol metabolism

As discussed herein, a putative effective amount may be for example a relative abundance of 0.001 (ie. 0.1%). It is important to note that relative abundance levels can be influenced by several factors. For example, a person with an E. coli infection at the time testing might show a zero for Parasutterella due to the fact that we are reporting relative abundance, and an overabundance of one type or several bacteria can drive down the abundance of bacteria normally making up a small portion of the microbiome.

While not wishing to be bound to a particular theory or hypothesis, the Inventors believe that “an effective amount” of Parasutterella in the gut microbiome of an individual is an absolute amount within the gut microbiome of the individual, that is, a sufficient number of Parasutterella such that the Parasutterella can exploit the gut microbiome modulating compound and such that LDL-cholesterol levels are lowered.

As will be apparent to one of skill in the art, an “effective amount” of a gut microbiome modulating compound is an amount that is believed to be sufficient to increase Parasutterella levels and improve at least one cardiovascular disease related measurement in the individual when administered on a dosage regimen or schedule over the suitable period of time. Such an effective amount will of course depend on the specific gut microbiome modulating compound being administered as well as other factors such as the age, weight, general condition and severity of symptoms in the individual.

As discussed herein, the prebiotic microbiome therapeutic may be resistant potato starch, delivered daily or as needed, for as long as the cardiovascular disease-related markers continue to show improvement compared to baseline levels.

As discussed herein, the effective amount of resistant potato starch may be for example 2 to 40 g, 2 to 30 g, 2 to 20 g, 5 to 40 g, 5 to 30 g, 5 to 20 g, or 10 to 20 g of resistant potato starch.

The effective amount may be administered in one or more doses during the day.

As used herein, “daily” does not necessarily mean “every day”, but may mean 9 out of 10 days, 8 out of 9 days, 7 out of 8 days, 6 out of 7 days, 5 out of 6 days, 4 out of 5 days, 3 out of 4 days, 2 out of 3 days, 1 out of 2 days, or combinations thereof.

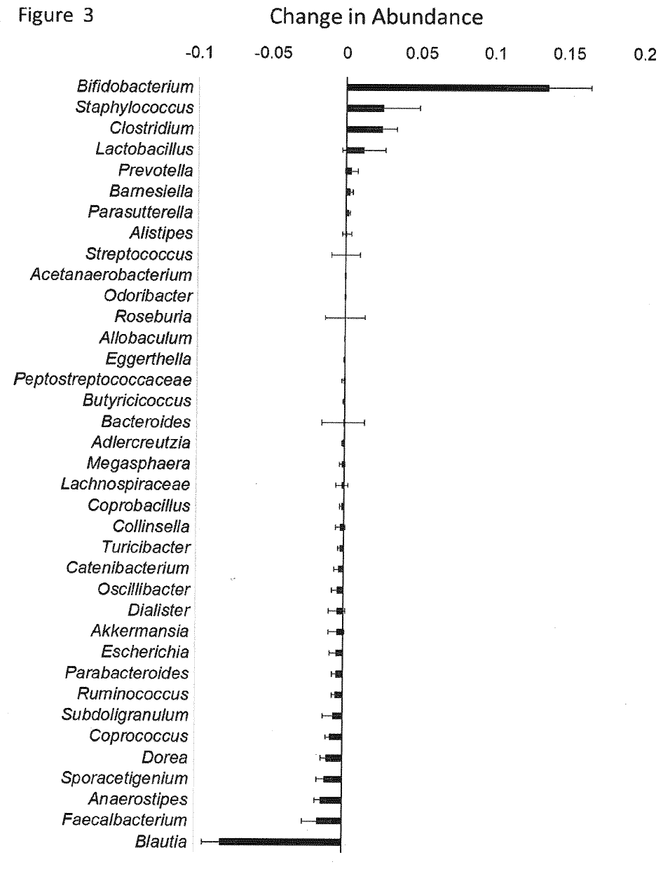

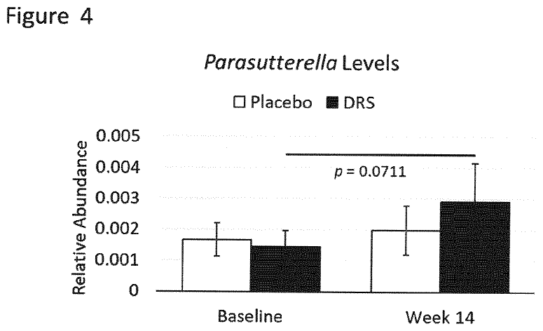

The connections between starch-fermenting Bifidobacterium, butyrate-producing members of the phylum Firmicutes, and health outcomes have been well-documented (De Vuyst and Leroy. 2011) but little research has been done to establish connections between other, more obscure bacteria in the gut microbiome. Furthermore, the role of Proteobacteria in the gut microbiome remains ambiguous, with evidence supporting both healthy (Scaldaferri et al. 2016) and pathogenic relationships (Gomes et al. 2016). The objective of this study was to explore connections between Parasutterella (phylum Proteobacteria) and various metabolic markers in prebiotic digestion resistant starch (DRS)-consuming individuals. Specifically, we measured correlations between improvements in LDL cholesterol and changes in bacterial genera of the gut microbiome in response to supplementation with DRS. DRS consumption led to increases in some genera of bacteria and decreases in others (FIG. 3). While we previously reported that DRS consumption led to reduced Escherichia coli levels (Alfa et al. 2018A), subsequent analysis revealed that Parasutterella was the only genus belonging to phylum Proteobacteria that increased in those consuming DRS (FIG. 3). This two-fold increase trended towards significance in DRS consumers (FIG. 4, p=0.0711) but placebo consumption had no effect on Parasutterella levels (FIG. 4, p=0.291).

We previously demonstrated that decreases in Sporacetigenium were correlated with improvements in blood glucose and insulin in those consuming DRS (Alfa et al. 2018B), so we asked whether increases in Parasutterella in response to DRS consumption were correlated with markers of cardiovascular and/or metabolic health. Pearson correlation coefficients (r) and p values were calculated for total cholesterol, triglycerides, low-density lipoprotein (LDL), high-density lipoprotein (HDL), blood glucose, and insulin levels. To control for multiple testing, the p values were then compared for significance using the Benjamini-Hochberg methods (Table 1). Increases in Parasutterella were significantly correlated with decreases in LDL levels in individuals consuming DRS (r=−0.400461; p=0.01284) but not with the other parameters. Notably, changes in Parasutterella were not significantly correlated with LDL levels in the placebo group (r=0.230647; p=0.1697).

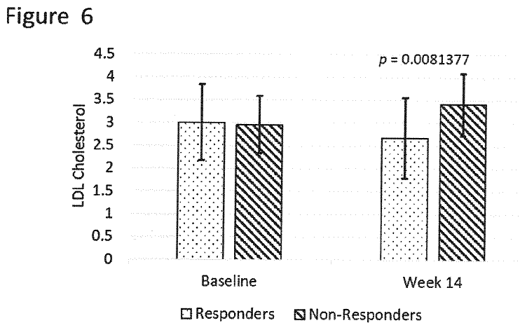

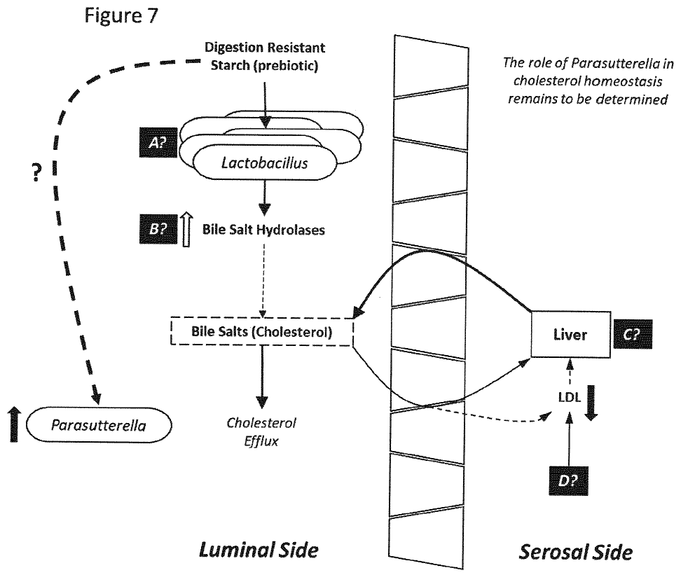

Despite the correlation between changes in Parasutterella and changes in LDL levels, DRS consumption did not lead to overall changes in LDL compared to placebo-consuming controls (Alfa et al. 2018A). In other words, only a subpopulation of DRS-consuming individuals experienced a change in LDL levels. Parasutterella is found in low abundance in the gut microbiome, suggesting that a minimum threshold may be required for DRS-mediated effects on LDL. We therefore asked whether baseline Parasutterella levels were higher in DRS consumers who experienced a decrease in LDL levels (Responders) compared to those in which LDL levels increased or were unchanged (Non-Responders). Consistent with this hypothesis, we found that Parasutterella levels were significantly more abundant in Responders compared to Non-Responders at both baseline and week 14 (FIG. 5). Of note, LDL cholesterol levels did not differ between Responders and Non-responders at baseline but there was a significant difference at 14 weeks (FIG. 6). Taken together, these data suggest that DRS consumption leads to reduced LDL levels in individuals with adequate levels of Parasutterella, that is, effective amounts of Parasutterella, in their gut microbiome.

Parasutterella (phylum Proteobacteria), a nonaerobic, Gram-negative, non-spore-forming coccobacillus, was originally described based on a strain isolated from a fecal sample from a healthy Japanese male (Nagai et al. 2009). Subsequent studies have failed to establish a consistent role for Parasutterella in human health (Chiodini et al. 2015, Ishaq et al 2017, Kreutzer et al. 2017, Chen et al. 2018A, Tang et al. 2018, Wang et al. 2018). Furthermore, prebiotic and probiotic supplementation has varying effects on Parasutterella levels (Metzler-Zebeli et al. 2015, Fang et al. 2017, Chen et al. 2018B, Cheng et al. 2018, Zhou et al. 2018).

Deep sea water (DSW) is one of several dietary supplements that tend to increase Parasutterella levels (Chen et al. 2017, Lin et al. 2017, Xie et al. 2017, Sun et al. 2018). Using a diet-induced hamster model of hypercholesterolemia, Lin and colleagues demonstrated that DSW also led to significant reductions in triglycerides, LDL, and total cholesterol, although Bacteroidetes was the only bacterial population significantly correlated with serum cholesterol and LDL in response to the high cholesterol diet (Lin et al. 2017). Increases in Parasutterella in response to GOS supplementation in mice were associated with significant reductions in triglycerides but not LDL levels (Cheng et al 2018). In obese humans, Parasutterella levels were inversely correlated with fat consumption but not total energy intake (Kreutzer et al. 2017). The Parasutterella type strain demonstrated esterase, leucine arylamidase, and arginine arylamidase activity, with weak caprylate esterase lipase and glycine arylamidase activity (Nagai et al. 2009), but the relationship between these activities, the metabolites they produce, and the effects on host LDL levels remain to be elucidated.

As discussed above, Ju et al demonstrated that Parasutterella levels can be dramatically increased via the addition of asparagine and aspartate to in vitro cultures, consistent with the predicted fermentation of these amino acids based on genomic analysis (Ju et al. 2019). While not wanting to be bound to a particular theory or hypothesis, the inventors note that potatoes are a natural source of both asparagine and aspartate, and that asparagine levels are highest in potato varieties used to produce resistant starches (Vivanti et al. 2006), suggesting that fermentation of asparagine and/or aspartate present in DRS like MSPrebiotic® may help lead to increases in Parasutterella in the gut microbiome. However, the utilization of asparagine and/or aspartate by Parasutterella does not explain the different LDL responses observed in Responders and Non-Responders because the amount of asparagine and aspartate available in the DRS would be comparable in both groups.

While Parasutterella is normally found at low levels in the mammalian gastrointestinal tract (Ju et al. 2019, Willing et al. 2010), it is important to note the significant dietary (ie. Diversity, dairy and meat consumption, etc.) and anatomical differences (ie. Cecum) between mice and humans, and that the effects of manipulating the gut microbiome in one specie do not necessarily translate to reasonable expectations of similar effects in other species.

It is known that Parasutterella is normally found at low levels in the mammalian gastrointestinal tract (Ju et al. 2019, Willing et al. 2010), Ju and others were only able to detect Parasutterella levels at 10⁶ CFU/g or higher in feces using real-time PCR (RT-PCR)-based methods. This distinction formed the threshold for considering animals to be ‘Parasutterella-free’ (aka. Control, or ‘CON’; Ju et al. 2019). Since Parasutterella is a core member of the gut microbiota, there were almost certainly low levels of Parasutterella (ie. less than 10⁶ CFU/g) in the control animals that went undetected by Ju and others using RT-PCR (Ju et al. 2019). Furthermore, the gut microbiomes belonging to animals having received probiotic Parasutterella supplementation (aka. ‘PARA’) had relative abundances (16S rRNA reads) of 0.64-1.88% (Ju et al. 2019), which are comparable to the average levels we observed in LDL Responders (0.2-0.4%). In this sense, CON animals are similar to the Non-Responders and PARA are similar to Responders, supporting our hypothesis that a threshold level of Parasutterella must be met that is be present in the gut microbiome in order to derive benefits that is beneficial cholesterol metabolism as described herein.

Interestingly, Ju and colleagues demonstrated that probiotic supplementation with Parasutterella led to reductions of bile salts in the gut, including cholic acid, taurocholic acid, taurodeoxycholic acid, 7-ketodeoxycholic acid, and glycolithocholic acid, with concomitant increases in taurine, consistent with the degradation of bile acids (Ju et al. 2019). Furthermore, the authors report increases in liver Cyp7a1 expression in PARA animals, encoding the rate-limiting enzyme for the production of cholic acid from cholesterol (Ju et al. 2019). It is important to note that the effect on total serum cholesterol in PARA animals was not statistically significant, and no data are reported relating to changes in LDL cholesterol (Ju et al. 2019). Also, it is unclear when the authors state “additionally, there was no significant difference in serum cholesterol between the CON and PARA group, which may be explained by the normal physiological state of the mice” whether they mean that Parasutterella supplementation may lead to significant improvements in serum cholesterol under diseased conditions where cholesterol levels are elevated, such as in hypercholesterolemia, or that the cholesterol differences are simply fluctuations within a normal physiological state (ie. The differences are ‘not real’; Ju et al. 2019).

As discussed herein, we propose that the surprising effect of Parasutterella on the host's physiology is dependent upon a variety of factors, including prebiotic consumption and the baseline composition of the host's gut microbiome (FIG. 7).

Other studies have demonstrated that differences in the gut microbiome can influence the output of physiologically important metabolites. For example, differences in the relative abundance of butyrogenic Eubaterium rectale were related to fecal butyrate levels in response to short-term consumption of raw potato starch (Venkataraman et al. 2016). While the metabolite(s) connecting Parasutterella to LDL production remain to be determined, it appears that individuals benefit from consuming DRS with respect to lipid levels when Parasutterella levels are higher, that is, above the threshold level or above the responder level. Furthermore, the relationship we describe here between DRS, Parasutterella, and LDL levels was observed in healthy participants, so subsequent studies using DRS in those with dyslipidemia may provide additional insight. The microbial ecosystem in our gut is incredibly complex and it is unlikely that a single microbe change alone could be responsible for a range of systemic health benefits. As our appreciation for the connections between diet, the gut microbiome, and human health grows, there will be opportunities to leverage this information in the context of personalized health.

While not wishing to be bound to a particular theory or hypothesis, Parasutterella, which ferments and metabolizes various peptides and amino acids, including asparagine and aspartate, may produce a specific metabolite or group of metabolites that communicate with the host's system to modulate internal transport of cholesterol and reduce LDL levels, such as the increase of Cyp7a1 expression in the liver.

Alternatively, Parasutterella may produce bile salt hydrolases (BSHs), which are known to cleave bile salts and prevent fat absorption by the host, reducing circulating levels of LDL. It is also possible that Parasutterella promotes the production of and/or stability of BSHs from other bacteria, which would have a similar lowering effect on LDL levels.

Taken together, the gut microbiome can positively influence cholesterol metabolism and influence cardiovascular disease (CVD) risk. Surprisingly, changes in Parasutterella levels at minimum serve as a marker for changes in the pre-CVD or CVD state. Specifically, it is believed that Parasutterella levels serve as a marker for CVD risk but may not be a driver of microbiome-related cholesterol metabolism. Accordingly, monitoring Parasutterella levels with at least one CVD-related parameter provides information on the effectiveness of gut microbiome related treatments. If Parasutterella levels increase in combination with improvement in one or more of the CVD-related parameters, this indicates that the individual can be treated using gut microbiome therapies. Alternatively, if Parasutterella levels increase but the CVD-related parameters do not improve, the CVD progression may be more heavily influenced by other factors, for example, genetic predisposition, diet, activity levels or the like and the gut microbiome modulating treatment should be stopped and replaced with more conventional treatments for CVD risk factors, like, for example, statins.

In summary, screening for Parasutterella levels in combination with CVD risk measures identifies those individuals who will benefit from positive modulation of the microbiome. The effectiveness of this strategy can then be measured by monitoring Parasutterella levels in combination with CVD risk measures. Our findings support these statements because changes in Parasutterella and changes in LDL cholesterol were inversely correlated in those consuming DRS but not the placebo and improvements in LDL levels when consuming DRS depended on a minimum level of Parasutterella.

This screen holds advantages over other microbiome-based CVD risk prediction methods. Overlapping and complementary roles of bacteria in the gut microbiome make it difficult to evaluate and base predictions on bacteria with known CVD risk-modifying functions like, for example, bile salt hydrolase (BSH) activity, which is a property of many probiotic bacteria, including many Lactobacillus species. In fact, Lactobacillus abundance was not correlated with LDL cholesterol levels in our data, highlighting the limitations of focusing on abundant bacteria. In comparison, Parasutterella makes up less than 0.5% of the gut microbiome, making this an important genus of low relative abundance. While the relationship between DRS and Parasutterella is unknown, and the mechanism by which DRS-mediated increases in Parasutterella translate to reduced LDL levels remain to be elucidated, the correlation between LDL improvement and Parasutterella increases in people consuming DRS provides a simple measure by which to confirm the efficacy of DRS treatment. Furthermore, our finding that Parasutterella levels were higher in responders (those whose LDL levels improved with DRS consumption) compared to non-responders means that co-administration of DRS with Parasutterella could convert non-responders to responders, thereby broadening the applicability of this screen. Increasing average Parasutterella levels from those in non-responders (˜0.05%) to those in responders (˜0.2%) is much more feasible for bacteria that exist at low levels of abundance because it is well known that the gut microbiome is refractory to large compositional changes via probiotic supplementation. Furthermore, Ju and colleagues demonstrate that colonization of the gut microbiome by Parasutterella does not significantly affect the overall composition of the microbiome (Ju et al. 2019). This is likely due to the fact that Parasutterella makes up a small total proportion of the microbiome and that its genome does not contain any virulence factors (Ju et al. 2019), so introduction of Parasutterella does not cause a domino effect by significantly competing with or complementing other extant members of the microbiome.

The complex heterogeneity of the human gut microbiome differs significantly from the human genome in that mutations in human genes affect highly-conserved physiological pathways shared by all people, while alterations in the microbiome may or may not influence the host's physiology, depending on the composition and redundancy if the resident microbes. In other words, screening the gut microbiome for markers linked to host physiology will help determine whether a person will respond to a treatment based on the unique composition of the gut microbiome. In this case, the microbiome-modulating therapy may or may not include supplementation with Parasutterella depending on baseline levels of this genus of bacteria. This provides a generic yet tailored method by which to test the efficacy of microbiome-based therapies for the lowering of LDL cholesterol and, by extension, improving cholesterol metabolism, lowering CVD risk factors, and decreasing the risk of a CVD event.

### Methods

### Clinical Study, Sample Collection, and Processing

Adults (aged 30-50 years and aged 70 years or older; 84 enrolled) consumed 30 g of placebo (digestible corn starch; Amioca TF, Ingredion, Brampton, ON) daily for two weeks before randomization to placebo or MSPrebiotic (digestion resistant potato starch (DRS); MSPrebiotics Inc., Carberry, MB) arms. Participants then consumed 30 g of placebo or DRS daily for 12 weeks (14 weeks total). Stool and fasting blood samples were collected at baseline and 14 weeks. Antibiotics alter the microbiome, so only samples from participants who did not receive antibiotic treatment within 5 weeks of stool sample collection were analyzed (N=75). Blood glucose and lipid (total cholesterol, triglycerides, low-density lipoprotein, high-density lipoprotein) levels were determined by Diagnostic Services Manitoba (Winnipeg, MB) and insulin levels by LipoScience Inc. (Raleigh, N.C.). Gut microbiome analysis was performed by 16S sequencing on the Illumina MiSeq platform and alignment as previously described (Alfa et al. 2018A, Schloss et al. 2009, Kozich et al. 2013). The data from all 84 participants (regardless of age) was pooled for this analysis.

### Statistical Analysis

Baseline values were subtracted from week 14 values and expressed as a change in percent (blood lipid, glucose, and insulin levels) or a change in relative abundance (bacteria) for each participant. Pearson's correlation coefficients (r) and p values for changes in Parasutterella and changes in blood measures were calculated and p values determined using Excel (Microsoft, Redmond, Wash.), as were Student's one-way t-Test calculations. We employed the Benjamini-Hochberg procedure (Benjamini and Hochberg. 1995) at a false discovery rate (FDR; q) of 0.1 to control for multiple testing during correlation analysis. The critical values for each parameter were generated by dividing the p value rank (i) by the total number of parameters analyzed (m), then multiplying this quotient by the FDR (q). p<0.05 was considered statistically significant.

### REFERENCES

Alfa, M J, Strang, D, P. Tappia, P S, Olson, N, DeGagne, P, Bray, D, Murray, B-L, and Hiebert, B. A Randomized Placebo Controlled Clinical Trial to Determine the Impact of Digestion Resistant Starch MSPrebiotic on Glucose, Insulin, and Insulin Resistance in Elderly and Mid-Age Adults, Fronteirs in Medicine (Lausanne), vol. 4, p. 260, 2018. A

Alfa, M J, Strang, D, Tappia, P S, Graham, M, Van Domselaar, G, Forbes, J D, Laminman, V, Olson, N, DeGagne, P, Bray, D, Murray, B-L, Dufault, B, and Lix, L M. A randomized trial to determine the impact of a digestion resistant, Clinical Nutrition, vol. 37, no. 3, pp. 797-807, 2018. B

Benjamini, Y, and Hochberg, Y. Controlling the False Discovery Rate: A Practical and Powerful Approach to Multiple Testing, Journal of the Royal Statistical Society. Series B (Methodological), vol. 57, no. 1, pp. 289-300, 1995.

Bush, J R, and Alfa, M J. Decreasing levels of Sporacetigenium correlate with improved diabetic parameters in healthy adults consuming MSPrebiotic® digestion resistant starch, JARCP, vol. 7, p. 176-180, 2018.

Cani, P. Human Gut Microbiome: Hopes, threats and promises. Gut, vol. 67, no. 9, pp. 1716-1725, 2018.

Chen, Y, Xiao, S, Gong, Z, Zhu, X, Yang, Q, Li, Y, Gao, S, Dong, Y, Shi, Z, Wang, Y, Weng, X, Li, Q, Cai, W, and Qiang, W. Wuji Wan Formula Ameliorates Diarrhea and Disordered Colonic Motility in Post-inflammation Irritable Bowel Syndrome Rats by Modulating the Gut Microbiota, Front Microbiol, vol. 8, p. 2307, 2017.

Chen, L, Li, S, Zheng, J, Li, W, Jiang, X, Zhao, X, Li, J, Che, L, Lin, Y, Xu, S, Feng, B, Fang, Z, and Wu, D. Effects of dietary Clostridium butyricum supplementation on growth performance, intestinal development, and immune response of weaned piglets challenged with lipopolysaccharide, J Anim Sci Biotechnol, vol. 9, p. 62, 2018. A.

Chen, Y, Wu, H, Wu, S, Lu, N, Wang, Y, Liu, H, Dong, L, Liu, T, and Shen, X. Parasutterella, in association with irritable bowel syndrome and intestinal chronic inflammation, J Gastroenterol Hepatol, vol. 33, no. 11, pp. 1844-1852, 2018. B.

Cheng, W, Lu, J, Lin, W, Wei, X, Li, H, Zhao, X, Jiang, A, and Yuan, J. Effects of a galacto-oligosaccharide-rich diet on fecal microbiota and metabolite profiles in mice, Food Funct, vol. 9, no. 3, pp. 1612-1620, 2018.

Chiodini, R, Dowd, S, Chamberlin, W, Galandiuk, S, Davis B, and Glassing, A. Microbial Population Differentials between Mucosal and Submucosal Intestinal Tissues in Advanced Crohn's Disease of the Ileum, PLoS ONE, vol. 10, no. 7, p. e0134382, 2015.

De Vuyst, L, and Leroy, F. Cross-feeding between bifidobacteria and butyrate-producing colon bacteria explains bifdobacterial competitiveness, butyrate production, and gas production, Int J Food Microbiol, vol. 149, no. 1, pp. 73-80, 2011.

Fang, D, Shi, D, Lu, L, Gu, S, Wu, W, Chen, Y, Guo, J, Li, A, Hu, X, Guo, F, Ye, J, Li, Y, and Li, L. Bifidobacterium pseudocatenulatum LI09 and Bifidobacterium catenulatum LI10 attenuate D-galactosamine-induced liver injury by modifying the gut microbiota, Sci Rep, vol. 7, no. 1, p. 8770, 2017.

Gibson, G, Hutkins, R, Sanders, M, Prescott, S, Reimer, R, Salminen, S, Scott, K, Stanton, C, Swanson, K, Cani, P, Verbeke K, and Reid, G. Expert consensus document: The International Scientific Association for Probiotics and Prebiotics (ISAPP) consensus statement on the definition and scope of prebiotics, Nat Rev Gastroenterol Hepatol, vol. 14, no. 8, pp. 491-502, 2017.

Gomes, T, Elias, W, Guth, SIB, Rodrigues, J, Piazza, R, Ferreira, L, and Martinez, M. Diarrheagenic Escherichia coli, Brazi J Microbiol, vol. 47, no. Suppl 1, pp. 3-30, 2016.

Ishaq, H, Mohammad, I, Guo, H, Shahzad, M, Hou, Y, Ma, C, Naseem, Z, Wu, X, Shi, P, and Xu, J. Molecular estimation of alteration in intestinal microbial composition in Hashimoto's thyroiditis patients, Biomed Pharmacother, vol. 95, pp. 865-874, 2017.

Ju, T, Yoon Kong, J, Stothard, P, Willing, B. Defining the role of Parasutterella, a previously uncharacterized member of the core gut microbiota, ISME J, vol.13, pp. 1520-1534, 2019.

Kozich, J J, Westcott, S L, Baxter, N T, Highlander, S K, and Schloss, P D. Development of a dual-index sequencing strategy and curation pipeline for analyzing amplicon sequence data on the MiSeq Illumina sequencing platform, Applied and Environmental Microbiology, vol. 79, no. 17, pp. 5112-20, 2013.

Kreutzer, C, Peters, S, Schulte, D, Fangmann, Turk, D, Wolff, K S, van Eimeren, T, Ahrens, M, Beckmann, J, Schafmayer, C, Becker, Kerby, T T, Rohr, A, Riedel, C, Heinsen, F, Degenhardt, F, Franke, A, Rosenstiel, P, Zubek, N, Henning, C, Freitag-Wolf, S, Dempfle, A, Psilopanagioti, A, Petrou-Papadaki, H, Lenk, L, Jansen, O, Schreiber, S, and Laudes M. Hypothalamic Inflammation in Human Obesity Is Mediated by Environmental and Genetic Factors, Diabetes, vol. 66, no. 9, pp. 2407-2415, 2017.

Lin, C, Chen, Y, Tsai, T, and Pan, T. Effects of deep sea water and Lactobacillus paracasei subsp. paracasei NTU 101 on hypercholesterolemia hamsters gut microbiota, Appl Microbial Biotechnol, vol. 101, no. 1, pp. 321-329, 2017.

Metzler-Zebeli, B, Schmitz-Esser, S, Mann, E, Grüll, D, Molnar, T, and Zebeli, Q. Adaptation of the cecal bacterial microbiome of growing pigs in response to resistant starch type 4, Appl Environ Microbiol, vol. 81, no. 24, pp. 8489-8499, 2015.

Nagai, F, Morotomi, M, Sakon, H, and Tanaka, R. Parasutterella excrementihominis gen. nov., sp. nov., a member of the family Alcaligenaceae isolated from human faeces, Int J Syst Evol Microbiol, vol. 59, no. Pt 7, pp. 1793-1797, 2009.

Scaldaferri, F, Gerardi, V, Mangiola, F, Lopetuso, L, Pizzoferrato, M, Petito, V, Papa, A, Stojanovic, J, Poscia, A, Cammarota, G, and Gasbarrini, A. Role and mechanisms of action of Escherichia coli Nissle 1917 in the maintenance of remission in ulcerative colitis patients: An update, World J Gastoenterol., vol. 22, no. 24, pp. 5505-5511, 2016.

Schloss, P D, Westcott, S L, Ryabin, T, Hall, J R, Hartmann, M, Hollister, E B, Lesniewski, R A, Oakley, B B, Parks, D H, Robinson, C J, Sahl, J W, Stres, B, Thallinger, G G, van Horn, D J, and Weber, C F. Introducing mothur: open-source, platform-independent, community-supported software for describing and comparing microbial communities, Applied and Environmental Microbiology, vol. 75, no. 23, pp. 7537-7541, 2009.

Sun, Y, Chen, S, Wei, R, Xie, X, Wang, C, Fan, S, Zhang, X, Su, J, Liu, J, Jia, W, and Wang, X. Metabolome and gut microbiota variation with long-term intake of Panax ginseng extracts on rats, Food Funct, vol. 9, no. 6, pp. 3547-3556, 2018.

Tang, R, Jiang, Y, Tan, A, Ye, J, Xian, X, Xie, Y, Wang, Q, Yao, Z, and Mo, Z. 16S rRNA gene sequencing reveals altered composition of gut microbiota in individuals with kidney stones, Urolithiasis, vol. 46, no. 6, pp. 503-514, 2018.

Venkataraman, A, Sieber, J, Schmidt, A, Waldron, C, Theis, K, and Schmidt, T. Variable responses of human microbiomes to dietary supplementation with resistant starch, Microbiome, vol. 4, no. 1, p. 33, 2016.

Vivanti, V, Finotti, E, and Friedman, M. Level of acrylamide precursors asparagine, fructose, glucose, and sucrose in potatoes sold at retail in Italy and the United States, JFS C: Food Chemistry and Toxicology, vol. 71, no. 2, pp. C81-C85, 2006.

Wang, X, Zhang, L, Wang, Y, Liu, X, Zhang, H, Liu, Y, Shen, N, Yang, J, and Gai, Z. Gut microbiota dysbiosis is associated with Henoch-Schönlein Purpura in children, Int Immunopharmacol, vol. 58, pp. 1-8, 2018.

Willing, B P, Dicksved, J, Halfvarson, J, Andersson, A F, Lucio, M, Zheng, Z, Järnerot, G, Tysk, C, Jansson, J K, and Engstrand, L. A pyrosequencing study in twins shows that gastrointestinal microbial profiles vary with inflammatory bowel disease phenotypes, Gastroenterology, vol. 139, no. 6, pp. 1844-1854, 2010. Xie, M, Chen, G, Wan, P, Dai, Z, Hu, B, Chen, L, Ou, S, Zeng, X, and Sun, Y. Modulating Effects of Dicaffeoylquinic Acids from Ilex kudingcha on Intestinal Microecology in Vitro, J Agric Food Chem, vol. 65, no. 47, pp. 10185-10196, 2017.

Zhou, W, Yan, Y, Mi, J, Zhang, H, Lu, L, Luo, Q, Li, X, Zeng, X, and Cao, Y. Simulated Digestion and Fermentation in Vitro by Human Gut Microbiota of Polysaccharides from Bee Collected Pollen of Chinese Wolfberry, J Agric Food Chem, vol. 66, no. 4, pp. 898-907, 2018.

TABLE 1
Correlations between the change in abundance of Parasutterella and changes in total
cholesterol, triglycerides, low-density lipoprotein (LDL), high-density lipoprotein
(HDL), blood glucose, and insulin levels in individuals consuming DRS.

| Health Parameter | r | p value | Rank (i) | [i/m]*q | Significant |
|---|---|---|---|---|---|
| LDL | −0.40046151 | 0.01284 | 1 | 0.016667 | Y |
| Blood Glucose | 0.256738899 | 0.119769 | 2 | 0.033333 | N |
| Cholesterol | −0.18932218 | 0.255775 | 3 | 0.05 | N |
| Insulin* | 0.130393557 | 0.448782 | 4 | 0.066667 | N |
| HDL | −0.06547371 | 0.698229 | 5 | 0.083333 | N |
| Triglycerides | 0.037206775 | 0.824522 | 6 | 0.1 | N |

The Benjamini-Hochberg method controls for false discovery of significant correlations (Benjamini and Hochberg. 1995). Results are rank ordered based on p value, and the p value is compared to the critical value ([i/m]*q, FDR=0.1) beginning with the lowest ranking parameter (Triglycerides). The first correlation with a p value lower than the critical value (LDL) and any higher-ranking correlations are considered significant. Positive Pearson correlation coefficient (r) values indicate positive correlations and negative r values indicate negative correlations. N=38 except for Insulin*, where N=36 due to fewer insulin measurements.

1-29. (canceled)

30. A method for reducing low-density lipid (LDL) cholesterol levels in an individual in need of such treatment comprising:
Detecting Parasutterella levels in a first gut microbiome sample from the individual at a first time point;
If the Parasutterella levels correspond to an effective amount of Parasutterella, administering to the individual a microbiome modulating treatment on a dosage regimen for a suitable period of time.

31. The method according to claim 30 wherein following the suitable period of time, at least one dyslipidemia related parameter measurement is taken.

32. The method according to claim 30 wherein following the suitable period of time: obtaining a second gut microbiome sample from the individual; detecting Parasutterella levels in the second sample; and comparing Parasutterella levels in the second gut microbiome sample to Parasutterella levels in the first gut microbiome sample, wherein if the Parasutterella levels are higher than in the first sample, continuing the dosage regimen for the individual.

33. The method according to claim 30 wherein the individual in need of such treatment is an individual with dyslipidemia or at risk of developing dyslipidemia.

34. The method according to claim 33 wherein the individual who is at risk of developing dyslipidemia is at risk based on genetic predisposition, familial history, heredity, lifestyle or on or more dyslipidemia or cardiovascular disease related parameters being evaluated.

35. The method according to claim 30 wherein the gut microbiome modulating compound is selected from the group consisting of: Resistant potato starch; probiotic genera, species, and strains; prebiotics supporting growth of probiotic genera, species, and strains; resistant starch from corn, tapioca, banana, grains, tubers and the like; resistant starch derived from high asparagine-containing varieties of potato, corn, tapioca, banana, grains, tubers and the like; resistant starch derived from high aspartate-containing varieties of potato, corn, tapioca, banana, grains, tubers and the like; fructooligosaccharides, galactooligosaccharides; xylooligosaccharides; mannanoligosaccharides; arabinoxylooligosaccharides; arabinogalactan polysaccharides; galactomannan polysaccharides; asparagine, asparagine-containing peptides or proteins, and carbohydrate-amino acid complexes that contain asparagine; aspartate, aspartate-containing peptides or proteins, and carbohydrate-amino acid complexes that contain aspartate.

36. The method according to claim 35 wherein the probiotic genera are selected from the group consisting of: Bifidobacterium; Staphylococcus; Clostridium; Lactobacillus; Prevotella; Barnsiella; Parasutterella; and combinations thereof.

37. The method according to claim 35 wherein the resistant starch is RS1, RS2, RS3, RS4, or RS5.

38. The method according to claim 30 wherein the microbiome modulating compound is selected from the group consisting of: Resistant potato starch; probiotic genera, species, and strains; prebiotics supporting growth of probiotic genera, species, and strains; resistant starch from corn, tapioca, banana, grains, tubers and the like; resistant starch derived from high asparagine-containing varieties of potato, corn, tapioca, banana, grains, tubers and the like; resistant starch derived from high aspartate-containing varieties of potato, corn, tapioca, banana, grains, tubers and the like; fructooligosaccharides, galactooligosaccharides; xylooligosaccharides; mannanoligosaccharides; arabinoxylooligosaccharides; arabinogalactan polysaccharides; galactomannan polysaccharides; asparagine, asparagine-containing peptides or proteins, and carbohydrate-amino acid complexes that contain asparagine; aspartate, aspartate-containing peptides or proteins, and carbohydrate-amino acid complexes that contain aspartate; dietary changes that support the growth of probiotic bacteria; dietary treatments that increase the availability of asparagine, aspartate, alanine, arginine, glycine, leucine, and/or other amino acids and/or other fermentation substrates to Parasutterella in the digestive tract; and antibiotics that target a bacterium/other bacteria that inhibit the growth of Parasutterella.

39. The method according to claim 30 wherein the microbiome modulating compound is selected from the group consisting of: Resistant potato starch; probiotic genera, species, and strains; prebiotics supporting growth of probiotic genera, species, and strains; resistant starch from corn, tapioca, banana, grains, tubers and the like; resistant starch derived from high asparagine-containing varieties of potato, corn, tapioca, banana, grains, tubers and the like; resistant starch derived from high aspartate-containing varieties of potato, corn, tapioca, banana, grains, tubers and the like; fructooligosaccharides, galactooligosaccharides; xylooligosaccharides; mannanoligosaccharides; arabinoxylooligosaccharides; arabinogalactan polysaccharides; galactomannan polysaccharides; asparagine, asparagine-containing peptides or proteins, and carbohydrate-amino acid complexes that contain asparagine; aspartate, aspartate-containing peptides or proteins, and carbohydrate-amino acid complexes that contain aspartate; dietary changes that support the growth of probiotic bacteria; dietary treatments that increase the availability of asparagine, aspartate, alanine, arginine, glycine, leucine, and/or other amino acids and/or other fermentation substrates to Parasutterella in the digestive tract; antibiotics that target a bacterium/other bacteria that inhibit the growth of Parasutterella; mixed plant cell wall fibers; beta-glucans; resistant dextrins; resistant maltodextrins; limit dextrins; polydextrose; alginate; pectin polysaccharides; hydroxypropylmethylcellulose; chitin; chondroitin-containing compounds; and glucosamine-containing compounds.

40. The method according to claim 39 wherein the mixed plant cell wall fibers comprise two or more of the following plant cell wall fibers in varying proportions: Cellulose, pectin, lignin, beta-glucan, and arabinoxylan regardless of source.

41. The method according to claim 31 wherein the dyslipidemia related parameter is selected from the group consisting of: Low density lipoprotein (LDL) levels; high density lipoprotein (HDL) levels; total cholesterol; total triglycerides; ratios involving LDL, HDL, total cholesterol, and/or triglycerides; systemic and/or tissue-specific inflammation markers; blood pressure; and metabolites.

42. The method according to claim 30 wherein the suitable period of time is from 1 week to 6 months.

43. The method according to claim 30 wherein Parasutterella levels are measured using a method selected from the group consisting of: Real-time polymerase chain reaction (RT-PCR)-based methods; quantitative PCR (qPCR)-based methods; microbiome sequencing; shotgun metagenomic sequencing; quantitative fluorescent in situ hybridization (FISH); antibody-based methods; and cell-binding based methods.

44. The method according to claim 30 wherein the gut microbiome modulating compound is resistant potato starch.

45. The method according to claim 44 wherein the effective amount is 2 to 40 g per day of resistant potato starch.

46. The method according to claim 45 wherein the effective amount may be administered in one or more doses during the day.

47. A method for converting a Parasutterella microbiome modulating treatment non-responder to a Parasutterella microbiome-modulating responder comprising:
Administering a Parasutterella increasing compound to an individual who has gut microbiome levels of Parasutterella below a responder threshold level on a dosage schedule or regimen.

48. The method according to claim 47 wherein the administration of the Parasutterella increasing compound continues until the gut microbiome levels of Parasutterella of the individual until the individual has an effective amount of gut microbiome Parasutterella.

49. The method according to claim 47 wherein the Parasutterella-increasing compounds are compounds known in the art that will effect dietary changes that increase the availability of alanine, arginine, glycine, leucine, and/or other amino acids and/or other fermentation substrates to Parasutterella in the digestive tract; or antibiotics or bacteriophage that target a bacterium/other bacteria that inhibit or limits the growth of Parasutterella.
